(19) United States
(12) Patent Application Publication
(10) Pub. No.: US 2025/0152472 A1
(43) Pub. Date: May 15, 2025

(54) HINGED BLINDING SHELL FOR HANGING A VIAL AND RELATED METHODS

(71) Applicant: Fisher Clinical Services Inc, Allentown, PA (US)

(72) Inventors: Michael Donschietz, Alburtis, PA (US); Harold Miller, Bethlehem, PA (US)

(21) Appl. No.: 19/025,424

(22) Filed: Jan. 16, 2025

Related U.S. Application Data
(63) Continuation of application No. 17/127,641, filed on Dec. 18, 2020.
(60) Provisional application No. 62/952,682, filed on Dec. 23, 2019.

(51) Int. Cl. A61J 1/14; A61J 1/16; A61M 5/14; B65D 23/08; B65D 75/22

(52) U.S. Cl. CPC A61J 1/1462; A61J 1/1406; A61J 1/16; A61M 5/1414; A61M 5/1417; A61M 2209/06; B65D 23/0885; B65D 75/22

(57) ABSTRACT

A vial blinding assembly includes a vial having a stopper with a needle penetrable septum that is aligned with a compartment of the vial. A blinding shell has an interior surface that bounds a cavity, the vial being disposed within the cavity. The blinding shell includes: an encircling sidewall extending between a first end and an opposing second end; a shield wall inwardly projecting from the first end of the sidewall and encircling an access opening, the access opening communicating with the cavity of the blinding shell and being aligned with the septum of the stopper; and a floor inwardly projecting from the second end of the sidewall so as to block access to the cavity at the second end, wherein the blinding shell comprises a first shell portion and a second shell portion that are hingedly coupled together. In one embodiment, a hanging tab outwardly projects from the floor of the blinding shall and has a mounting hole extending therethrough.

### CROSS-REFERENCE TO RELATED APPLICATIONS

This continuation application claims priority to the currently pending U.S. patent application Ser. No. 17/127,641 having the filing date of Dec. 18, 2020, which itself claims the benefit of U.S. Provisional Application No. 62/952,682, filed Dec. 23, 2019, which is incorporated herein by specific reference.

### BACKGROUND

### 1. Technical Field

The present disclosure relates to blinding shells that can be used to cover vials during blinded studies of therapeutic drugs and to related methods.

### 2. The Relevant Technology

A large number of therapeutic drugs are provided in liquid form and are administered intravenously. Such drugs are commonly housed within a bag or vial. Dispensing of the drug intravenously typically requires that the bag or vial be suspended in an inverted orientation so that the port or septum through which the drug is accessed is facing downwardly. This orientation enables the drug to freely flow under the force of gravity when an IV line is coupled thereto.

Blinded testing of a drug during clinical trials requires that the drug be shielded so that the drug cannot be analyzed by the patient or the person administering the drug. Blinding shields are commonly used on vials where the vials are configured to receive a needle from a syringe to withdraw the therapeutic drug for administering. However, because of the complexity of vials that need to be suspended in an inverted orientation for intravenous delivery, blinding shells have not been developed for such vials.

Furthermore, a shortcoming of many conventional blinding shells is that different sized blinding shells are typically configured to house different sized vials. The problem is that if it is necessary to switch vial configurations or if it is necessary to concurrently send multiple vials for use on a patient, where the vials are different configurations, the patient and/or person administering may potentially notice the different configurations of blinding shells that are used to enclose the different sized vials. In turn, the patient and/or person administering may deduce that, because there are differences in the configurations of the blinding shells, the trial product contained therein has changed or has certain properties, thereby dimensioning the efficacy of the blinded trial.

Accordingly, what is needed are blinding shields and assemblies that overcome all or some of the above shortcomings and other problems known in the art.

### SUMMARY OF THE DISCLOSURE

In a first independent aspect of the disclosure, a vial blinding assembly includes:
a vial comprising:
   a bottle having a neck that bounds an inlet opening to a chamber of the bottle;
   a stopper disposed on the bottle so as to cover the inlet opening, the stopper comprising a needle penetrable septum aligned within the inlet opening; and
   a retainer securing the stopper to the bottle;
a blinding shell having an interior surface that bounds a cavity, the vial being disposed within the cavity, the blinding shell comprising:
   an encircling sidewall extending between a first end and an opposing second end;
   a shield wall inwardly projecting from the first end of the sidewall and encircling an access opening, the access opening communicating with the cavity of the blinding shell and being aligned with the septum of the stopper, the shield wall at least partially covering the retainer or the stopper; and
   a floor inwardly projecting from the second end of the sidewall so as to block access to the cavity at the second end;
wherein the blinding shell comprises a first shell portion and a second shell portion that are hingedly coupled together.

In one embodiment, the retainer includes an inwardly projecting flange that encircles an aperture that is aligned with the septum, the shield wall extending over at least a portion of the flange so that the access opening is aligned with the aperture.

In another embodiment, the flange has an outside face and the shield wall covers at least 80%, 85%, 90%, 95%, or 97% of the outside face of the flange.

In another embodiment, the blinding shell covers all of the vial except for the septum.

In another embodiment, the blinding shell covers all of the vial except for what can be seen through the access opening.

In another embodiment, the access opening has a maximum diameter that is less than 10 mm, 7 mm, 5 mm, or 3 mm.

In another embodiment, the access opening is circular.

Another embodiment includes means for securing the first shell portion to the second shell portion so that the vial is captured within the cavity of the blinding shell.

In another embodiment, the means for securing comprises a projection extending from the first shell portion that is press fit into a coupling hole formed on the second shell portion.

In another embodiment, the coupling hole comprise a blind pocket.

In another embodiment, the blinding shell is comprised of a plastic.

In another embodiment, the blinding shell is opaque.

In another embodiment, the blinding shell has an exterior surface, a distance between the interior surface and the exterior surface being less than 3 mm, 1 mm or 0.5 mm.

In another embodiment, the first shell portion and the second shell portion are hingedly coupled together by a hinge.

In another embodiment, the hinge comprises a living hinge that is integrally formed as a unitary member with the first shell portion and the second shell portion.

In another embodiment, a hanging tab outwardly projects from the floor, the hanging tab having a mounting hole laterally extending therethrough.

In another embodiment, the hanging tab outwardly projects along a central longitudinal axis of the cavity.

In another embodiment, the encircling sidewall comprises:
an annular body extending between a first end an opposing second end, the floor being disposed at the second end of the body; and
an annular neck inwardly constricting from the first end of the body to the shield wall.

In another embodiment, a flexible seal extends between the first shell portion and a second shell portion and is secured thereto by an adhesive.

Another embodiment further includes:
a first closure tab outwardly projecting from a first side of the neck; and
   a second closure tab outwardly projecting from a second side of the neck, the second side of the neck being opposite the first side.

In another embodiment, the first closure tab and the second closure tab are disposed within a common plane.

In another embodiment, the first closure tab comprises:
a first closure tab portion projecting from the neck and forming a portion of the first shell portion; and
a second closure tab portion projecting from the neck and forming a portion of the second shell portion.

Another embodiment includes:
a projection extending from the first closure tab portion; and
a coupling hole formed on the second closure tab portion, the projection being press fit into the coupling hole so as to form a secure friction fit therein.

Another embodiment includes a flexible seal extending between the first closure tab portion and the second closure tab portion and being secured thereto by an adhesive.

Another embodiment includes:
the first shell portion comprising:
   a first body portion extending between a first end and an opposing second end, the first body portion being a portion of the body and bounding a portion of the cavity;
   a first neck portion projecting from the first end of the first body portion, the first neck portion being a portion of the neck and bounding a portion of the cavity;
   a first shield wall portion inwardly projecting from first neck portion, the first shield wall portion being a portion of the neck and bounding a portion of the cavity;
   a first floor portion inwardly projecting from the second end of the first body portion, the first floor portion being a portion of the floor and bounding a portion of the cavity; and
   a first closure tab portion outwardly projecting from the first neck portion.

In another embodiment, a hanging tab outwardly projects from the first floor portion, a mounting hole laterally extending through the hanging tab.

Another embodiment includes:
the second shell portion comprising:
   a second body portion extending between a first end and an opposing second end, the second body portion being a portion of the body and bounding a portion of the cavity;
   a second neck portion projecting from the first end of the second body portion, the second neck portion being a portion of the neck and bounding a portion of the cavity;
   a second shield wall portion inwardly projecting from second neck portion, the second shield wall portion being a portion of the neck and bounding a portion of the cavity;
   a second shield wall portion inwardly projecting from second neck portion, the second shield wall portion being a portion of the neck and bounding a portion of the cavity;
   a second floor portion inwardly projecting from the second end of the second body portion, the second floor portion being a portion of the floor and bounding a portion of the cavity; and
   a second closure tab portion outwardly projecting from the second neck portion and being disposed against the first closure tab.

Another embodiment includes:
a projection extending from the first closure tab portion or the second closure tab portion; and
a coupling hole formed on the other of the first closure tab portion or the second closure tab portion, the projection being press fit into the coupling hole so as to form a secure friction fit therein.

In a second independent aspect of the disclosure, a vial blinding assembly includes:
a blinding shell having an interior surface that bounds a cavity, the cavity being configured to receive a vial, the blinding shell comprising:
   an encircling sidewall extending between a first and an opposing second end;
   a shield wall inwardly projecting from the first end of the sidewall and encircling an access opening, the access opening communicating with the cavity of the blinding shell;
   a floor inwardly projecting from the second end of the sidewall so as to block access to the cavity at the second end; and
   a hanging tab outwardly projecting from the floor, the hanging tab having a mounting hole laterally extending therethrough;
wherein the blinding shell comprises a first shell portion and a second shell portion that are hingedly coupled together.

In another embodiment, the hanging tab outwardly projects along a central longitudinal axis of the cavity.

In another embodiment, the hanging tab is planar.

In another embodiment, the hanging tab extends between a first side of the sidewall and an opposing second side of the sidewall.

In another embodiment, the hanging tab comprises:
a first hanging tab portion comprising a portion of the first shell portion, the mounting hole extending through the first hanging tab portion;
a second hanging tab port on comprising a portion of the second shell portion, the mounting hole extending though the second hanging tab portion, the second hanging tab portion being disposed against the first hanging tab portion.

In another embodiment, a vial is disposed within the cavity of the blinding shell.

In another embodiment, the blinding shell covers all of the vial except for what can be seen through the access opening.

In another embodiment, means are provided for securing the first shell portion to the second shell portion so that a vial can be captured within the cavity of the blinding shell.

In another embodiment, the first shell portion and the second shell portion are hingedly coupled together by a hinge, the hinge comprising a living hinge that is integrally formed as a unitary member with the first shell portion and the second shell portion.

A third independent aspect of the disclosure includes a method for using a vial blinding assembly, the method comprising:
positioning a vial within a first cavity portion of a first shell portion, a second shell portion being hingedly coupled to the first shell portion, the vial comprising:
   a bottle having a neck that bounds an inlet opening to a chamber of the bottle;
   a stopper disposed on the bottle so as to cover the inlet opening, the stopper comprising a needle penetrable septum aligned within the inlet opening;
   and a retainer securing the stopper to the bottle; and
moving at least one of the first shell portion or the second shell portion so that the vial is captured between the first shell portion and the second shell portion, the first shell portion and the second shell portion at least partially covering the retainer or the stopper and bounding an access opening that communicates with the septum.

In another embodiment, moving at least one of the first shell portion or the second shell portion comprises a projection forming a portion of the first shell portion being press fit into a coupling hole formed on the second shell portion so that a secure friction fit connection is formed therebetween.

In another embodiment, the coupling hole expands as the projection is received therein.

In another embodiment, the first shell portion and the second shell portion cover all of the vial except for what can be seen through the access opening.

Another embodiment includes shipping the first shell portion and the second shell portion with the vial captured therebetween as part of a blinded study of a liquid trial product disposed within the vial.

In a fourth independent aspect of the disclosure, a method for a vial blinding assembly includes:
positioning a vial within a first cavity portion of a first shell portion, a second shell portion being hingedly coupled to the first shell portion, the vial comprising:
   a bottle having a neck that bounds an inlet opening to a chamber of the bottle;
   a stopper disposed on the bottle so as to cover the inlet opening, the stopper comprising a needle penetrable septum aligned within the inlet opening; and
   a retainer securing the stopper to the bottle;
moving the second shell portion relative to the first shell portion so that the vial is captured between the first shell portion and the second shell portion, the first shell portion and the second shell portion forming at least a portion of a blinding shell, the blinding shell having a first end with an access opening that communicates with the septum and an opposing second end with an outwardly projecting hanging tab formed thereat, a lateral mounting hole passing through the hanging tab.

Another embodiment includes, suspending the blinding shell by the hanging tab so that the vial is inverted with the septum facing downward.

Another embodiment includes, coupling the hanging tab to an IV stand.

Another embodiment includes, passing a needle through the septum so that the needle communicates with a liquid trial product disposed within the vial, an IV line being fluid coupled with the needle.

Another embodiment includes, administering the liquid trial product to a patient intravenously as part of a blinded study of the liquid trial product.

Another embodiment includes, moving at least one of the first shell portion or the second shell portion comprises a projection forming a portion of the first shell portion being press fit into a coupling hole formed on the second shell portion so that a secure friction fit connection is formed therebetween.

In another embodiment, the blinding shell covers all of the vial except for what can be seen through the access opening.

In a fifth independent aspect of a disclosure, a kit includes:
a first vial blinding assembly comprising:
   a first blinding shell having an interior surface that bounds a first cavity and an exterior surface, an access opening passing through the first blinding shell so as to communicate with the first cavity; and
   a first vial having a chamber with a liquid trial product disposed therein, the chamber being sealed closed by a stopper having a needle penetrable septum, the first vial being disposed within the first cavity of the first blinding shell so that the septum of the first vial is aligned with the access opening of the first blinding shell;
a second vial blinding assembly comprising:
   a second blinding shell having an interior surface that bounds a second cavity and an exterior surface, an access opening passing through the second blinding shell so as to communicate with the second cavity; and
   a second vial having a chamber with a liquid trial product disposed therein, the chamber being sealed closed by a stopper having a needle penetrable septum, the second vial being disposed within the second cavity of the second blinding shell so that the septum of the second vial is aligned with the access opening of the second blinding shell,
wherein the first cavity of the first blinding shell has a different configuration than the second cavity of the second blinding shell but the exterior surface of the first blinding shell has the same configuration as the exterior surface of the second blinding shell.

In another embodiment, the first vial has a different configuration than the second vial.

In another embodiment, the first blinding shell and the second blinding shell are opaque.

Another embodiment includes:
the first blinding shell covers all of the first vial except for what can be seen through the access opening of the first blinding shell; and
the second blinding shell covers all of the second vial except for what can be seen through the access opening of the second blinding shell.

In a sixth independent aspect of the disclosure includes, a method includes:
enclosing a first vial within a first cavity of a first blinding shell so that an access opening passing through the first blinding shell is aligned with a needle penetrable septum of the first vial, the first vial housing a liquid trial product; and
enclosing a second vial within a second cavity of a second blinding shell so that an access opening passing through the second blinding shell is aligned with a needle penetrable septum of the second vial, the second vial housing a liquid trial product, the first cavity of the first blinding shell having a different configuration than the second cavity of the second blinding shell but an exterior surface of the first blinding shell having the same configuration as an entire exterior surface of the second blinding shell.

In another embodiment, the first vial has a different configuration than the second vial.

In another embodiment, the first blinding shell and the second blinding shell are opaque.

In another embodiment:
the first blinding shell covers all of the first vial except for what can be seen through the access opening of the first blinding shell; and
the second blinding shell covers all of the second vial except for what can be seen through the access opening of the second blinding shell.

Another embodiment includes, delivering the first blinding shell housing the first vial and the second blinding shell housing the second vial to a location for administering the liquid trial product in a blinded study.

Each of the above independent aspects may include any of the features, options and possibilities set out in this document, including those under each of the above independent aspects.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a perspective view of a vial blinding assembly coupled to an IV line;

FIG. 2 is a perspective top view of a blinding shell of the vial blinding assembly shown in FIG. 1 in an open position;

FIG. 3 is a perspective bottom view of the blinding shell shown in FIG. 2;

Figure is a perspective view of the blinding shell shown in FIG. 2 having a vial disposed therein;

FIG. 5 is an elevated cross-sectional view of the vial shown in FIG. 4;

FIG. 6 is a perspective view of the blinding shell shown in FIG. 4 being moved to a closed position with the vial disposed therein;

FIG. 7 is an elevated front, cross sectional view of two adjacently disposed blinding shells having cavities of different configurations;

FIG. 8 is an elevated front view of the blinding shells shown in FIG. 7;

FIG. 9 is an elevated front view of adjacently disposed vials having different configurations that are configured to be received within the cavities of the blinding shells shown in FIG. 7;

FIG. 10 is a perspective view of an alternative embodiment of a blinding shell wherein the hanging tab has been removed; and

FIG. 11 is a perspective view of the blinding shell shown in FIG. 10 in a closed position.

### DETAILED DESCRIPTION OF THE PREFERRED EMBODIMENTS

Before describing the present disclosure in detail, it is to be understood that this disclosure is not limited to parameters of the particularly exemplified systems, methods, apparatus, products, processes, compositions, and/or kits, which may, of course, vary. It is also to be understood that the terminology used herein is only for the purpose of describing particular embodiments of the present disclosure, and is not necessarily intended to limit the scope of the disclosure in any particular manner. Thus, while the present disclosure will be described in detail with reference to specific embodiments, features, aspects, configurations, etc., the descriptions are illustrative and are not to be construed as limiting the scope of the claimed invention. Various modifications can be made to the illustrated embodiments, features, aspects, configurations, etc. without departing from the spirit and scope of the invention as defined by the claims. Thus, while various aspects and embodiments have been disclosed herein, other aspects and embodiments are contemplated.

Unless defined otherwise, all technical and scientific terms used herein have the same meaning as commonly understood by one of ordinary skill in the art to which the present disclosure pertains. While a number of methods and materials similar or equivalent to those described herein can be used in the practice of the present disclosure, only certain exemplary materials and methods are described herein.

Various aspects of the present disclosure, including devices, systems, methods, etc., may be illustrated with reference to one or more exemplary embodiments or implementations. As used herein, the terms “alternative embodiment” and/or “exemplary implementation” means “serving as an example, instance, or illustration,” and should not necessarily be construed as preferred or advantageous over other embodiments or implementations disclosed herein. In addition, reference to one or more embodiments is intended to provide illustrative examples without limiting the scope of the invention, which is indicated by the appended claims rather than by the following description.

It will be noted that, as used in this specification and the appended claims, the singular forms “a,” “an” and “the” include plural referents unless the content clearly dictates otherwise. Thus, for example, reference to an “insert” includes one, two, or more inserts. As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

Various aspects of the present disclosure can be illustrated by describing components that are coupled, attached, connected, and/or joined together. As used herein, the terms “coupled”, “attached”, “connected,” and/or “joined” are used to indicate either a direct connection between two components or, where appropriate, an indirect connection to one another through intervening or intermediate components. In contrast, when a component is referred to as being “directly coupled”, “directly attached”, “directly connected,” and/or “directly joined” to another component, no intervening elements are present or contemplated. Thus, as used herein, the terms “connection,” “connected,” and the like do not necessarily imply direct contact between the two or more elements. In addition, components that are coupled, attached, connected, and/or joined together are not necessarily (reversibly or permanently) secured to one another.

As used herein, directional and/or arbitrary terms, such as “top,” “bottom,” “front,” “back,” “left,” “right,” “up,” “down,” “upper,” “lower,” “inner,” “outer,” “internal,” “external,” “interior,” “exterior,” “proximal,” “distal” and the like can be used solely to indicate relative directions and/or orientations and may not otherwise be intended to limit the scope of the disclosure, including the specification, invention, and/or claims.

Where possible, like numbering of elements have been used in various figures. In addition, similar elements and/or elements having similar functions may be designated by similar numbering (e.g., element “10” and element “210.”) Furthermore, alternative configurations of a particular element may each include separate letters appended to the element number. Accordingly, an appended letter can be used to designate an alternative design, structure, function, implementation, and/or embodiment of an element or feature without an appended letter. Similarly, multiple instances of an element and or sub-elements of a parent element may each include separate letters appended to the element number. In each case, the element label may be used without an appended letter to generally refer to instances of the element or any one of the alternative elements. Element labels including an appended letter can be used to refer to a specific instance of the element or to distinguish or draw attention to multiple uses of the element. However, element labels including an appended letter are not meant to be limited to the specific and/or particular embodiment(s) in which they are illustrated. In other words, reference to a specific feature in relation to one embodiment should not be construed as being limited to applications only within said embodiment.

It will also be appreciated that where a range of values (e.g., less than, greater than, at least, and/or up to a certain value, and/or between two recited values) is disclosed or recited, any specific value or range of values falling within the disclosed range of values is likewise disclosed and contemplated herein. Thus, disclosure of an illustrative measurement or distance less than or equal to about 10 units or between 0 and 10 units includes, illustratively, a specific disclosure of: (i) a measurement of 9 units, 5 units, 1 units, or any other value between 0 and 10 units, including 0 units and/or 10 units; and/or (ii) a measurement between 9 units and 1 units, between 8 units and 2 units, between 6 units and 4 units, and/or any other range of values between 0 and 10 units.

It is also noted that systems, methods, apparatus, devices, products, processes, compositions, and/or kits, etc., according to certain embodiments of the present disclosure may include, incorporate, or otherwise comprise properties, features, aspects, steps, components, members, and/or elements described in other embodiments disclosed and/or described herein. Thus, reference to a specific feature, aspect, steps, component, member, element, etc. in relation to one embodiment should not be construed as being limited to applications only within said embodiment. In addition, reference to a specific benefit, advantage, problem, solution, method of use, etc. in relation to one embodiment should not be construed as being limited to applications only within said embodiment.

The headings used herein are for organizational purposes only and are not meant to be used to limit the scope of the description or the claims. To facilitate understanding, like reference numerals have been used, where possible, to designate like elements common to the figures.

The present disclosure is directed to vial blinding assemblies used in blinded studies of therapeutic drugs and related methods and kits. In general, the vial blinding assemblies include a vial holding a liquid trial product and a blinding shell that is opaque and selectively covers the vial. The blinding shell can be hinged and can include a mounting tab projecting from a floor thereof to enable the vial to be suspended in an inverted orientation, such as on an IV stand. By suspending the vial inverted, the liquid trial product can be administered to a patient intravenously under the force of gravity. The blinding shell functions to help preclude or limit those receiving or administering the liquid trial product from the vial from detecting any properties of the vial or the liquid trial product contained therein so that the fidelity and efficacy of the blinded study is maintained.

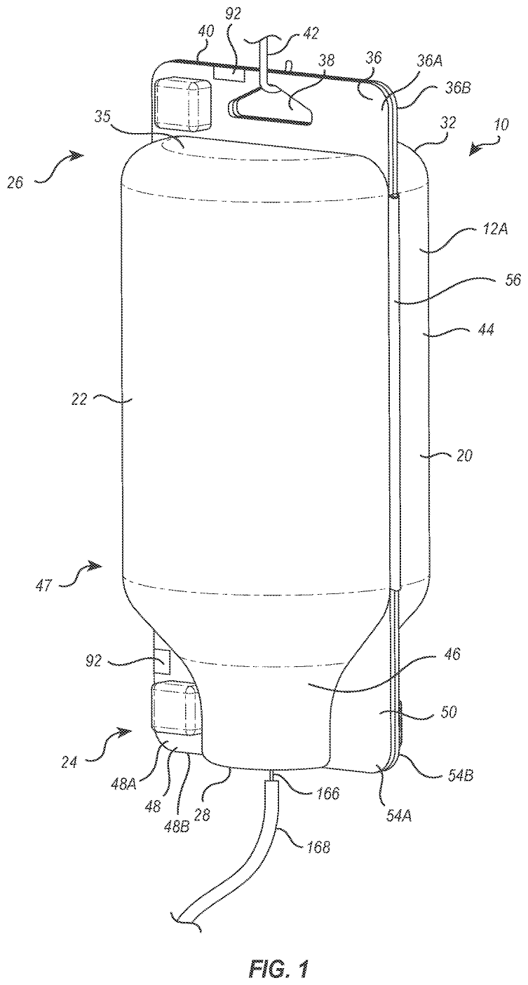

Depicted in FIG. 1 is one embodiment of an inventive vial blinding assembly 10 incorporating features of the present disclosure. Vial blinding assembly 10 includes a blinding shell 12A and a vial 14 (FIG. 4) that is disposed therein. More specifically, with reference to FIGS. 1 and 4, blinding shell 12 has an interior surface 16 that bounds a cavity 18 and has an opposing exterior surface 20. Cavity 18 has a central longitudinal axis 21 extending therethrough. During use, vial 14 is disposed within cavity 18.

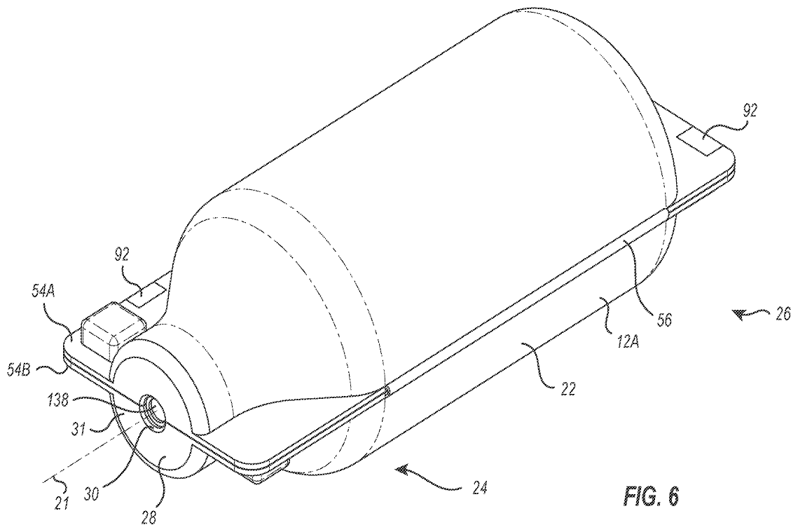

Described in further detail, blinding shell 12A, as shown in FIG. 1, has an encircling sidewall 22 that extends between a first end 24 and an opposing second end 26. As shown in FIG. 6, a shield wall 28 inwardly projects from first end 24 of sidewall 22 and centrally encircles an access opening 30 that communicates with cavity 18. In the embodiment depicted, access opening 30 is circular and has a diameter being at least or less than 3 mm, 5 mm, 7 mm, 10 mm, 15 mm, or is in a range between any two of the foregoing. In other embodiments, access opening 30 can be elliptical, polygonal, irregular, or have other configurations. Shield wall 28 has an interior surface 29 (FIG. 4) that bounds a portion of cavity 18 and an opposing exterior surface 31 (FIG. 6) that is openly exposed. In the embodiment depicted, shield wall 28 and at least a portion interior surface 29 and exterior surface 31 are disposed in planes that orthogonally intersect with central longitudinal axis 21. This configuration enables a close tolerance fit with vial 14 and simplifies production of blinding shell 12A. However, other configurations can also be used.

Returning to FIG. 1, a floor 32 inwardly projects from second end 26 of sidewall 22 so as to completely block access to or otherwise cover cavity 18 at second end 26. As depicted in FIGS. 1 and 4, floor 32 has an inside face 34 and an opposing outside face 35. As will be discussed below in greater detail, the thickness of floor 32 extending between faces 34 and 35 can vary depending on the size of vial 14 being housed within cavity 18. In the embodiment depicted, floor 32 and at least a portion of inside face 34 and outside face 35 are disposed in planes that orthogonally intersect with central longitudinal axis 21. This configuration enables a close tolerance fit with vial 14 and simplifies production of blinding shell 12A. However, other configurations can also be used.

As shown in FIG. 1, a hanging tab 36 outwardly projects from floor 32 and has a mounting hole 38 laterally extending therethrough More specifically, hanging tab 36 projects outward along the longitudinal direction of blinding shell 12A and typically outwardly projects along central longitudinal axis 21 (FIG. 4). Hanging tab 36 also extends laterally toward or to opposing sides of floor 32. Although not required, in one embodiment, hanging tab 36 is planar and is disposed within a plane that is parallel to central longitudinal axis 21 and/or is orthogonal to floor 32/outside face 35. Hanging tab 36 has a height extending from floor 32 to a top edge 40. To accommodate mounting hole 38, the height of hanging tab 36 is typically at least 5 mm, 7 mm, 10 mm, 15 mm, or 20 mm or is in a range between any two of the foregoing values. Other dimensions can also be used.

Mounting hole 38 is configured to receive a hanger so as to enable suspending of vial blinding assembly 10. For example, a hanger 42 can be attached to or be part of an IV stand for suspending vial blinding assembly 10 from the IV stand. Hanger 42 can be a hook, loop, strap, bar, rod, hanger or other structure that can be received within mounting hole 38. Mounting hole 38 can be triangular, circular, polygonal or have other configurations. Commonly, mounting hole 38 will have a maximum diameter that is greater than 4 mm, 6 mm, 8 mm, 13 mm, or 18 mm or is in a range between any two of the foregoing values. Other dimensions can also be used.

Sidewall 22 can be further defined as comprising an annular body 44 that extends between a first end 47 and second end 26. At least a portion of body 44 typically has a cylindrical configuration and bounds or encircles a portion of cavity 18. Sidewall 22 also includes an annular neck 46 that inwardly constricts from first end 47 of body 44 to first end 24/shield wall 28. Neck 46 also bounds or encircles a portion of cavity 18. Outwardly projecting from opposing sides of neck 46 is a first closure tab 48 and an opposing second closure tab 50. Closure tabs 48 and 50 are typically planer and extend along the length of neck 46 from body 44 to shield wall 28. However, it is not necessary that closure tabs 48 and 50 extend along the full length of neck 46. Closure tabs 48 and 50 are typically disposed in a common plane and are typically disposed in a plane that is common with hanging tab 36 and/or passes through central longitudinal axis 21.

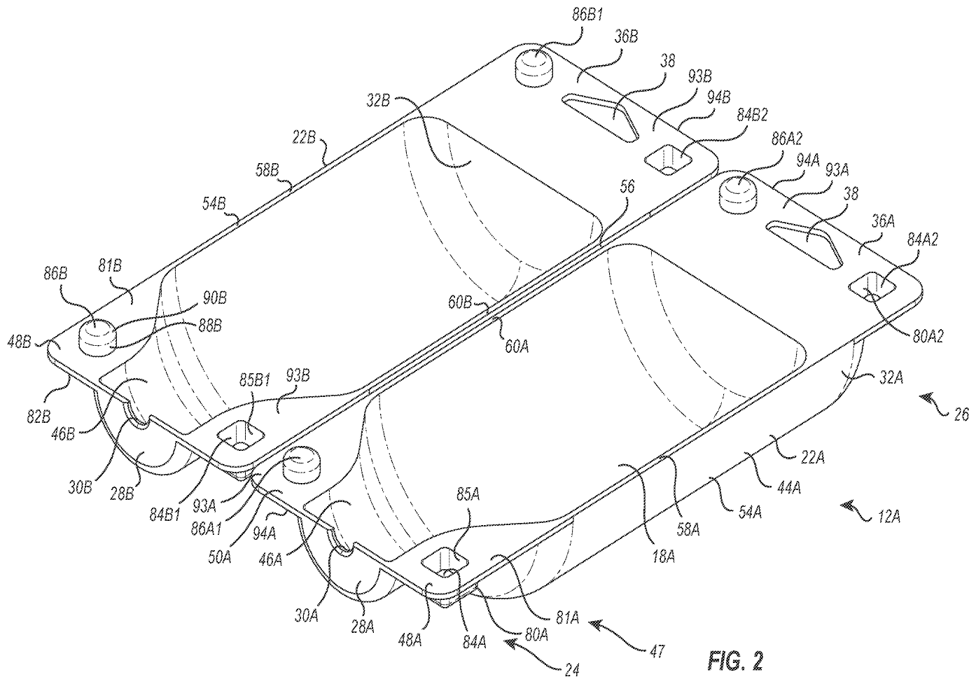

As depicted in FIG. 2, blinding shell 12A can also be described as comprising a first shell portion 54A, a second shell portion 54B, and a hinge 56 extending therebetween. Shell portion 54A comprises a sidewall portion 22A that extends between first end 24 and opposing second end 26 and extends between an outside edge 58A and an inside edge 60A. Sidewall portion 22A partially bounds a cavity portion 18A. Inwardly extending from first end 24 of sidewall portion 22A is a shield wall portion 28A partially bounding a notch 30A. Shield wall portion 28A also partially bounds cavity portion 18A. A floor portion 32A inwardly projects from second end 26 of sidewall portion 22A so as to partially bound cavity portion 18A thereat. A hanging tab portion 36A outwardly projects from floor portion 32A and has mounting hole 38 laterally passing therethrough. Hanging tab portion 36A can be have the same configuration, orientation and dimensions as previously discussed with regard to hanging tab 36.

Sidewall portion 22A can also be defined as comprising a body portion 44A that extends from first end 47 to second end 26 and that bounds a portion of cavity portion 18A. A neck portion 46A extends from first end 47 to shield wall portion 28A and inwardly constricts as it extends therebetween. Outwardly projecting from opposing sides of neck portion 46A is a closure tab portion 48A and a closure tab portion 50A. Closure tab portions 48A and 50A can be planar and disposed within a common plane.

Shell portion 54B can have the same or substantially the same configuration as shell portion 54A. As such, the same discussion and elements as discussed above with regard to shell portion 54A are likewise applicable to shell portion 54B. Furthermore, like elements between shell portion 54A and 54B are identified by like reference characters except that the reference characters of shell portion 54B include the suffix “B.” In general, each of shell portions 54A and 54B form equal halves of blinding shell 12A. Thus, when shell portions 54A and 54B are coupled together in a closed position, as discussed below in further detail, sidewall portions 22A and 22B combine to form sidewall 22; cavity portions 18A and 18B combine to form cavity 18; shield wall portions 28A and 28B combine to form shield wall 28; notches 30A and 30B combine to form access opening 30; floor portions 32A and 32B combine to form floor 32; hanging tab portions 36A and 36B combine to form hanging tab 36; body portions 44A and 44B combine to form body 44; neck portions 46A and 46B combine to form neck 46; closure tab portions 48A and 48B combine to form closure tab 48; and closure tab portions 50A and 50B combine to form closure tab 50. Furthermore, because shell portions 54A and 54B form equal halves of blinding shell 12A, the prior discussions with regard to sidewall 22, cavity 18, shield wall 28, access opening 30, floor 32, hanging tab 36, body 44, neck 46, closure tab 48, and closure tab 50 is also applicant to their related portions unless self-evident otherwise.

Hinge 56 extends between inside edges 60A and 60B and enables shell portions 54A and 54B to move between an open position, as shown in FIG. 2, where vial 14 can be inserted into or removed from blinding shell 12A, and a closed position, as shown in FIG. 1, wherein vial 14 is captured between shell portions 54A and 54B. In one embodiment, hinge 56 comprises a living hinge that is integrally formed as a single, continuation, unitary member with shell portions 54A and 54B. That is, in one embodiment, blinding shell 12A is formed from a plastic material through a molding process, such as injection molding. Hinge 56 can be formed by producing the mold so that a thin, flexible piece of plastic extends between inside edges 60A and 60B. In an alternative embodiment, hinge 56 can be separately formed from shell portions 54A and 54B and then attached thereto, such as by welding, adhesive, crimping, or mechanical attachment. For example, hinge 56 can comprise a tape, adhesive strip, or a flexible sheet or film that is secured to and extends between inside edges 60A and 60B. In still other embodiments, hinge 56 can be eliminated and shell portions 54A and 54B can be formed as two separate and discrete members that are coupled together without the use of a hinge.

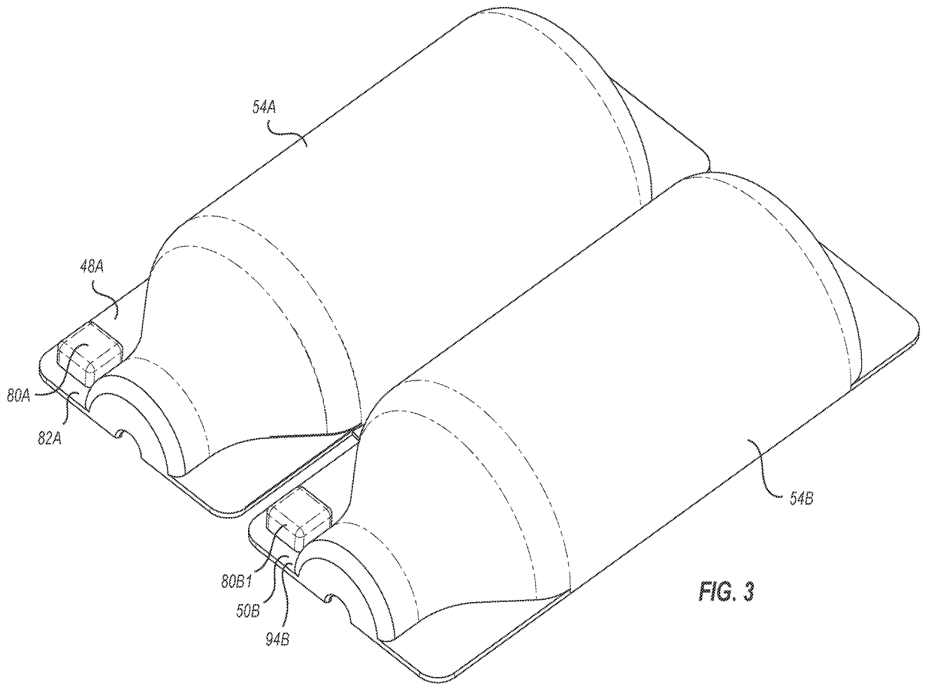

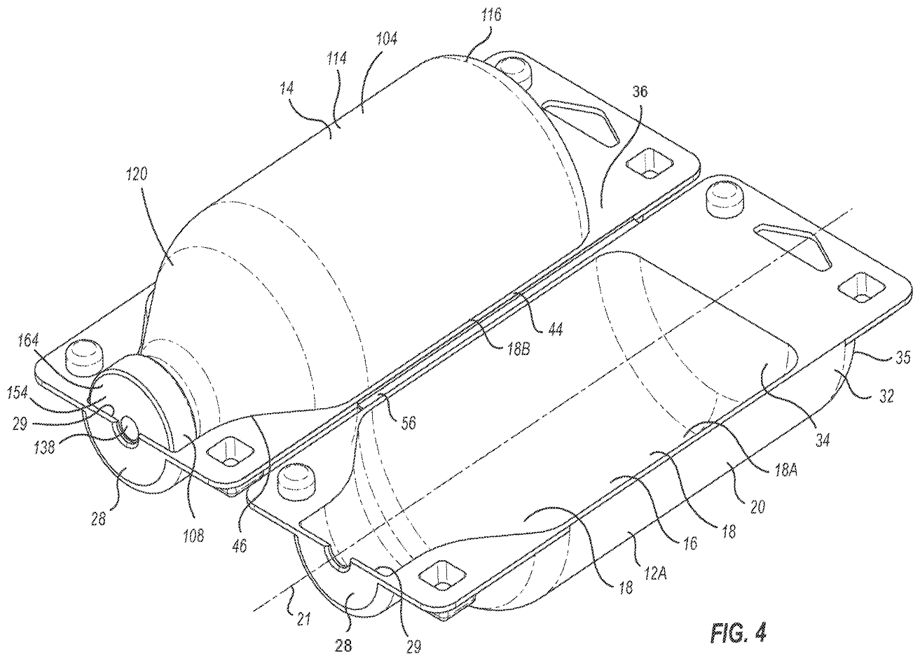

In one embodiment of the present disclosure, means are also provided for securing first shell portion 54A to second shell portion 54B in the closed position so that vial 14 is captured therebetween. By way of example and not by limitation, as depicted in FIGS. 2 and 3, closure tab portion 48A has an interior surface 81A and an opposing exterior surfaced 82A. An enclosure 80A outwardly projects from exterior surface 82A. A coupling hole 84A is formed on interior surface 81A and projects into enclosure 80A so as to be partially bounded by enclosure 80A. Coupling hole 84A is this bounded by an interior surface 85A that is partially formed from closure tab portion 48A and partially formed from enclosure 80A. In this embodiment, coupling hole 84A is in the form of a blind pocket. In other embodiments, however, coupling hole 84A can pass all the way through enclosure 80A and thus would not be blind. In the embodiment depicted, coupling hole 84A has a square or rectangular transverse cross section. As discussed below in more detail, in other embodiments, the transverse cross section of coupling hole 84A can have other configurations such as circular, polygonal or irregular.

Closure tab portion 48B of second shell portion 54B also has an interior surface 81B and an opposing exterior surface 82B. A projection 86B outwardly projects from interior surface 81B. In the embodiment depicted, projection 86B has a cylindrical body 88B that upstands from interior surface 81B and extends to a rounded perimeter edge 90B. Projection 86B is configured so that when blinding shell 12A is folded toward the closed position, projection 86B aligns with coupling hole 84A. Furthermore, projection 86B is configured relative to coupling hole 84A so that a manual press fit connection can be made between projection 86B and coupling hole 84A. Specifically, the maximum diameter of projection 86B is slightly larger than the height and/or width of coupling hole 84A. As such, as blinding shell 12A is folded together toward the closed position so that projection 86B aligned with coupling hole 84A, projection 86B can be manually press fit into coupling hole 84A so as to move blinding shell 12A into the fully closed position. Pressing projection 86B into coupling hole 84A forces interior surface 85A of coupling hole 84A to outwardly flex or bend so as to form a secure friction fit between projection 86B and interior surface 85A of coupling hole 84A. Rounded perimeter edge 90B of projection 86B enables easy alignment and insertion of projection 86B into coupling hole 84A.

In the present embodiment, the connection of projection 86B into coupling hole 84A does not make a permanent connection. That is, projection 86B can be removed from coupling hole 84A by applying sufficient manual force. However, the press fit connection is sufficient to prevent unintended removal of projection 86B from coupling hole 84A, i.e., prevent unintended movement of blinding shell 12 from the closed position to the open position. However, in some embodiments, pressing projection 86B into coupling hole 84A can produce at least some plastic deformation of interior surface 85A of coupling hole 84A, thereby providing some permanent and visible indication of an initial closing. In other embodiments, the connection between projection 86B and coupling hole 84A can be a permanent connection, i.e., cannot be separated without some permanent deformation or destruction of a portion of blinding shell 12A. For example, this can be accomplished by using an adhesive between projection 86B and coupling hole 84A. In other embodiments, a rib, barb, or other mechanical structure can be formed on projection 86B that mates with a corresponding structure on coupling hole 84A so as to produce a permanent connection between the two members.

It is appreciated that the corresponding sizes and shapes of projection 86B and coupling hole 84A can be modified into a variety of different configurations, i.e., sizes and shapes, that still produce the desired friction fit connection when projection 86B is received within coupling hole 84A. For example, coupling hole 84A could have a circular transvers cross section while projection 86B has a polygonal transverse cross section, such as octagonal with rounded perimeter edge 90B, that is sized so that the desired friction fit connection is formed when the projection is received within the coupling hole. Other complementary configurations can also be used. Although not always required, projection 86B is commonly formed as a solid member, i.e., no pockets or cavities formed thereon, so that projection 86B does not collapse or expand while being pressed into coupling hole 84A.

As depicted in FIG. 2, a plurality of spaced apart projections 86 can be used with corresponding coupling holes 84 to secure shell portion 54A and 54B together at opposing ends 24 and 26. For example, closure tab portion 50A has an interior surface 93A and an opposing exterior surface 94A. A projection 86A1 outwardly projects from interior surface 93A. Likewise, closure tab portion 50B has an enclosure 80B1 extending from exterior surface 94B. A coupling hole 84B1 is formed on interior surface 93B and projects into enclosure 80B1 so as to be partially bounded by enclosure 80A. Projection 86A1 and coupling hole 84B1 function the same as projection 86B and coupling hole 84A discussed above. That is, as blinding shell 12A is moved into the closed position, projection 86A1 can be pressed into coupling hole 84B1 so as to form a secure, friction tight, press fit connection between projection 86A1 and an interior surface 85B1 of coupling hole 84B1. The alternatives and configurations discussed above with regard to projection 86B and coupling hole 84A are also applicable to projection 86A1 and coupling hole 84B1, respectively.

Furthermore, hanging tab portion 36A has an interior surface 93A and an opposing exterior surface 94A while hanging tab portion 36B has an interior surface 93B and an opposing exterior surface 94B. A projection 86A2 outwardly projects from interior surface 93A on one side of mounting hole 38 while a coupling hole 84A2 extends through interior surface 93A an into an enclosure 80A2 on the opposing side of mounting hole 38. Projection 86A2 and coupling hole 84A2 are configured to make the same press-fit connection with a coupling hole 84B2 and a projection 86B2 that are formed on interior surface 93B of hanging tab portion 36B on opposing sides of mounting hole 38 when shell portions 54A and 54B are moved to the closed position Again, all of the previously discussion alternatives with regard to the projections and coupling holes can be used. It is appreciated that shell portions 54A and 54B can be formed with 1, 2, 3, 4, 5, 6, or more combinations of spaced apart projections 86 and coupling holes 84. The number of projections 86 and coupling holes 84 used is in part dependent upon the size of vial 14 and, in turn, the size of blinding shell 12A. One goal is to have sufficient projections 86 and coupling holes 84 to ensure that there is no accidental separation between shell portions 54A and 54B and to help ensure that shell portions 54A and 54B cannot be easily flexed or bent to enable peeking at vial 14 between shell portions 54A and 54B. In other embodiments, each of projections 86 and coupling holes 84 can be switched between the different shell portions 54A and 54B or all of projection 86 can be formed on one shell portion 54 while all of the coupling holes are formed on the other shell portion 54.

Once blinding shell 12A is moved to the closed position and projections 86 are received within coupling holes 84, one, two, three, or more seals 92, as shown in FIG. 1, can be secured to blinding shell 12A that extend between shell portions 54A and 54B. Seals 92 can comprise a piece of tape, sticker, or other flexible strip that can be secured by an adhesive. Seals 92 are preferably configured so that they cannot be removed without tearing. The one or more seals 92 can be spaced apart and ensure that once that once blinding shell 12A has been moved to the closed position, it has not subsequently been moved to the open position. For example, in one embodiment, one or more seal 92 can be placed so as to extend between closure tab portions 48A and 48B and/or one or more seals 92 can be placed so as to extend between hanging tab portions 36A and 36B. Seals 92 can also prevent peeking between shell portions 54A and 54B. As such, seals 92 further ensuring the efficacy and fidelity of the blinded study.

In other alternative embodiments, a variety of other structures and techniques can be used for securing first shell portion 54A to second shell portion 54B in the closed position so that vial 14 is captured therebetween. By way of example and not by limitation, projections 86 and coupling holes 84 can be eliminated and be replaced with adhesive, adhesive strips, or other types of fasteners such as clamps, crimps, screws, or the like.

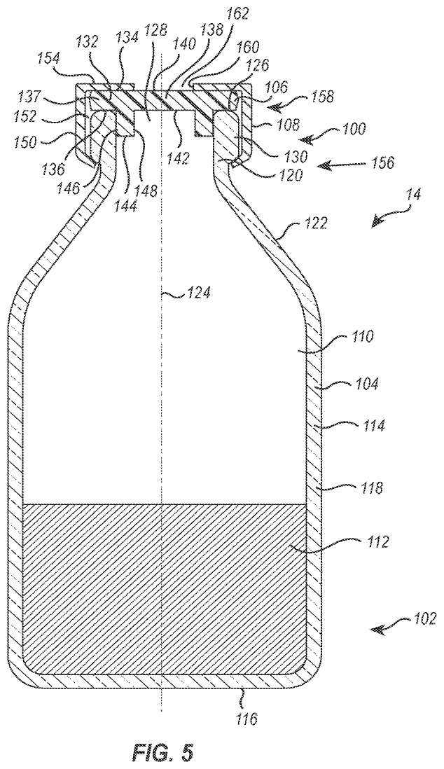

Turning to FIG. 4, with blinding shell 12A in the open position, vial 14 can be positioned within one of cavity portions 18A or 18B As depicted in FIG. 5, vial 14 has an upper end 100 and an opposing lower end 102. Vial 14 includes a bottle 104 having a stopper 106 disposed thereon Stopper 106 is secured in place by a retainer 108. More specifically, bottle 104 bounds a chamber 110 that is configured to hold a liquid trial product 112. As used herein, a “liquid trial product” refers to a liquid product being used in a blinded clinical trial and can include an active drug product that is under investigation, a related placebo product, a control product, or a comparator product.

Bottle 104 includes a body 114 having a circular floor 116 with cylindrical sidewall 118 upstanding therefrom. Bottle 60 also includes a neck 120 that is constricted relative to sidewall 118. An annular shoulder 122 inwardly slopes from an upper end of sidewall 118 to neck 120. Shoulder 122 inwardly slopes at an angle relative to a central longitudinal axis 124 of bottle 104. Neck 120 terminates at an end face 126. End face 126 encircles an inlet opening 128 that communicates with chamber 110. An annular flange 130 radially outwardly projects from annular neck 120 adjacent to end face 126. Bottle 104 is typically made of glass or serializable plastic and is commonly transparent.

With continued reference to FIG. 5, stopper 106 comprises an annular top 132 having a top surface 134 and an opposing bottom surface 136. Opposing surfaces 134 and 136 can be planar and disposed in parallel alignment. However, some contours can also be disposed on one or both surfaces. Top 132 can further be defined as comprising an annular rim portion 137 that encircles a central septum 138. Septum 138 has a top surface 140 and an opposing bottom surface 142 and is needle penetrable.

Stopper 106 further comprises tubular stem 144 that projects from bottom surface 136 of top 132. Tubular stem 144 has an exterior surface 146 and an opposing interior surface 148. Interior surface 148 encircles bottom surface 142 of septum 138. Stopper 106 is typically formed a single, integral, unitary member, as opposed to two or more members connected together, and is typically made from a rubber or elastomeric material that can produced a liquid tight seal with bottle 104. The material for stopper 106 is also selected so that a needle of a syringe can effectively pass through septum 138 for withdrawing a portion of liquid trial product 112 that is housed within bottle 104 and is self-sealing once the needle is withdrawn.

During assembly, stem 144 is advanced into inlet opening 128 until rim portion 137 comes to rest on top of end face 126 of neck 120. In this configuration, exterior surface 146 of stem 144 sits against the interior surface of neck 120 to help effect a seal therebetween. Bottom surface 142 of septum 138 is aligned with and communicates with chamber 110.

Stopper 106 is secured to bottle 104 by retainer 108. Retainer 108 comprises a circular collar 150 having a first end 156 and an opposing second end 158. Collar 150 encircles a cavity 152 that is configured to receive stopper 106 and at least a portion of neck 120 of bottle 104. Retainer 108 further comprises an annular flange 154 that radially inwardly projects from second end 158 of collar 150. Flange 154 terminates an inner face 160 that encircles an aperture 162. Retainer 64 is generally formed from a bendable material, such as a metal, and is typically formed from aluminum.

During assembly, stopper 106 is positioned on bottle 104 so as to cover inlet opening 128, as discussed above. Retainer 108 is then advanced over stopper 106. Specifically, collar 150 is advanced over stopper 106 and over flange 130 of bottle 104 until flange 154 of retainer 108 rests on top of stopper 106 and first end 156 of collar 150 extends below flange 130 of bottle 104. Retainer 108 is then crimped onto bottle 104 by radially inwardly bending first end 156 of collar 150 below flange 130 of bottle 104. This crimping of retainer 108 functions to compress stopper 106 against end face 126 of bottle 104 to produce a liquid tight seal therebetween and also prevents manual separation of retainer 108 and stopper 106 from bottle 104 without the use of a tool or at least partial destruction of retainer 108. The crimping of retainer 108 is typically achieved by a crimper that is passed over retainer 108 and then manipulated to radially inwardly compress first end 156 of collar 150. Such crimpers are known in the art.

In the assembled configuration, top surface 140 of septum 138 is openly exposed through aperture 162 of retainer 108. When it is desired to access liquid trial product 112 within vial 14, a needle can be passed through septum 138 and into chamber 110 of bottle 104 to withdraw a portion of liquid trial product 112.

It is appreciated that retainer 108 can have a variety of different configurations and can be used in a variety of different ways and still function to secure stopper 106 to bottle 104. By way of example and not by limitation, in contrast to being crimped onto bottle 104, retainer 108 could be formed to produce a snap-fit connection with bottle 104 or could be formed in two halves that are clamped together about neck 120. In still other embodiments, the retainer could be attached by adhesive, press fit, or constriction under heating, e.g., shrink wrapping. It is also appreciated that stopper 106 and vial 14 can have different configurations, i.e., sizes and shapes.

As previously noted, in general, vial 14 is configured to fit within cavity 18 of blinding shell 12A so that vial 14 is housed or captured within cavity 18 of blinding shell 12A when blinding shell 12A is moved into the closed position. More specifically, vial 14 typically has a configured that is generally complementary to cavity 18 so that there is minimal movement or play of vial 14 relative to blinding shell 12A when blinding shell 12A is in the closed position. For example, with reference to FIG. 4, when blinding shell 12A is in the closed position, floor 116 of bottle 104 is typically disposed against or adjacent to floor 32 of blinding shell 12A; body 114 of bottle 104 is typically disposed against or adjacent to the interior surface of body 44 of blinding shell 12A; and neck of 120 of bottle 104 is typically disposed against or adjacent to the interior surface of neck 46 of blinding shell 12A; and flange 154 of retainer 108 is typically disposed against or adjacent to interior surface 29 of shield wall 28. Cavity 18 is typically sized relative to vial 14 so that when vial 14 is enclosed within blinding shell 12A, vial 14 can only move longitudinally within cavity 18 by less than 10 mm and more commonly less than 7 mm, 5 mm or 3 mm. Furthermore, cavity 18 is typically sized relative to vial 14 so that when vial 14 is enclosed within blinding shell 12A, retainer 108 and/or vial 14 can only move laterally within cavity 18 by less than 6 mm and more commonly less 4 mm or 2 mm. Where cavity 18 is larger than vial 14, an insert can be positioned within cavity to occupy at least a portion of the open spaced within cavity 18. For example, the insert could be in the form of a disk that is positioned between floor 116 of bottle 104 and floor 32 of blinding shell 12A. In another embodiment, the insert could be in the form of a cup that receives lower end 102 of vial 14. Other configurations of inserts can also be used.

As previously discussed, one of the objectives of blinding shell 12A is to cover vial 14 as part of a blinded study so that the patient receiving the liquid trial product 112 and/or the person administering the liquid trial product 112 are unable to see vial 14 or liquid trial product 112. The coving of vial 14 limits the ability of the patient and/or person administering to deduce or attempt to deduce any properties or changes in liquid trial product 112, thereby improving the efficacy and fidelity of the blinded study. To that end blinding shell 12A is opaque. This can be accomplished by either forming blinding shell 12A from an opaque material or by applying an opaque coating or covering, such as a paint or printing, or an opaque layer, such as an adhesive sticker covering. Other approaches can also be used so that blinding shell 12A is blacked out. In one embodiment, blinding shell 12A is molded or otherwise formed from a plastic such as Acrylonitrile Butadiene Styrene (ABS). ABS is an opaque thermoplastic and amorphous polymer. Other plastics/polymers can also be used. In still other embodiments, blinding shell 12A can be formed from a metal, fiberglass, composite or the like.

It is typically preferred that blinding shell 12A cover as much of vial 14 as possible while still permitting withdrawal of liquid trial product 112 through septum 138. Accordingly, as shown in FIGS. 4 and 6, in one embodiment blinding shell 12A is configured so that when blinding shell 12A is in the closed position with vial 14 disposed therein, the only portion of vial 14 that can be seen is that which can be seen through access opening 30 of blinding shell 12A. For example, blinding shell 12A is configured so that when blinding shell 12A is in the closed position and vial 14 is captured therein, shield wall 28 extends over a portion of stopper 106 (FIG. 5) and retainer 108. More specifically, shield wall 28 extends over/covers a portion of flange 154 of retainer 108. In another embodiment, flange 154 has an outside face 164. Shield wall 28 extends over/covers all of outside face 164 of flange 154 or extends over/covers at least 80%, 85%, 90%, 95% or 97% of outside face 164 of flange 154 or extends over/cover a range between any two of the foregoing values. Furthermore, access opening 30 of shield wall 28 is aligned with septum 138 so that septum 138 can be accessed through access opening 30 when blinding shell 12A is in the closed position. Shield wall 28 may cover none of septum 138 or may cover a portion of septum 138 but does not cover all of septum 138. In another embodiment, blinding shell 12A is configured so that when blinding shell 12A is in the closed position within vial 14 disposed therein, the only portion of vial 14 that can be seen when shield wall 28 is viewed straight on is septum 138, a portion of septum 138, or septum 138 in combination with less than 10%, 5% or 2% of the surface area of outside face 164 of flange 154.

To facilitate use of blinding shell 12A in a blinded study, blinding shell 12A is moved to the open position, as shown in FIG. 4, and vial 14 having liquid trial product 112 therein is positioned within one of cavity portions 18A or 18B or is otherwise disposed between the cavity portions 18A and 18B. Blinding shell 12A is then moved to the closed position as shown in FIG. 6, and discussed above, so that vial 14 is captured within blinding shell 12A with septum 138 or substantially only septum 138, such as discussed above, being visible through access opening 30. One or more seals 92 can then be positioned that extend between shell portions 54A and 54B so as to prevent opening blinding shell 12A. In one embodiment, the blinding shells 12A and vials 14 can be shipped to a medical facility or administering person, such as a physician or nurse, in a disassembled state. The medical facility or administering person can then enclose vials 14 within blinding shells 12A prior to administering the liquid trial product 112 to the patient. In other embodiments, such as where it is desired that the administering person does not see vials 14, the blinding shells 12A and vials 14 can be preassembled into vial blinding assemblies 10 and then shipped or otherwise transported to a patient, medical facility, or administering person for subsequent administering.

During administering of liquid trial product 112, vial blinding assembly 10/blinding shell 12/vial 14 can be suspended in an inverted position so that septum 138 of vial 14 and shield wall 28 are facing downward and hanging tab 36 is projecting upward. In one method of suspending, hanger 42 or some other structure is secured to hanging tab 36 by passing through mounting hole 38 and is then connected to a stand, such as an IV stand, or some other support structure. In other methods, a portion of the stand, such as an IV stand, or other support structure forms hanger 42 and is directly passed through mounting hole 38 to suspend vial blinding assembly 10/blinding shell 12/vial 14 in the inverted position. In yet other embodiments, clamps, clips, or other fasteners can be used to couple hanging tab 36, with or without passing through mounting hole 38, to a stand or other supporting structure. As such, in some embodiments, mounting hole 38 can be eliminated from hanging tab 36.

Either prior to or after suspending blinding assembly 10/blinding shell 12/vial 14 in the inverted position, a needle 166 connected to an IV line 168 is passed through access opening 30 and exposed septum 138 so as to access liquid trial product 112. The IV line 168 can then be fluid coupled to a patient using conventional methods for intravenous delivery of liquid trial product 112 to the patient under the force of gravity.

Although vial 14 is disposed within blinding shell 12 that is specifically adapted for suspending vial 14 in an inverted orientation, it is not necessary that liquid trial product 112 be delivered intravenously. For example, liquid trial product 112 can also be dispensed through a syringe by simply passing the needle of the syringe through septum 138, drawing a portion of liquid trial product 112 into the syringe through the needle, withdrawing the needle from septum 138, and then delivering the withdrawn liquid trial product 112 to the patient by either injecting the liquid trial product 112 into the patient using the syringe or using other conventional delivery methods. Where a syringe is being used, it is not necessary to suspend blinding assembly 10/blinding shell 12A; vial 14 in an inverted position.

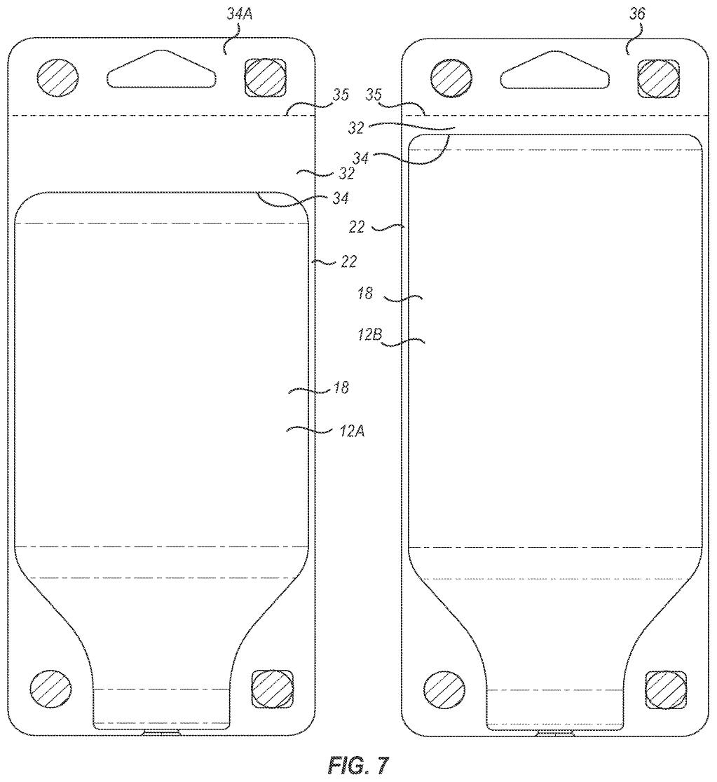

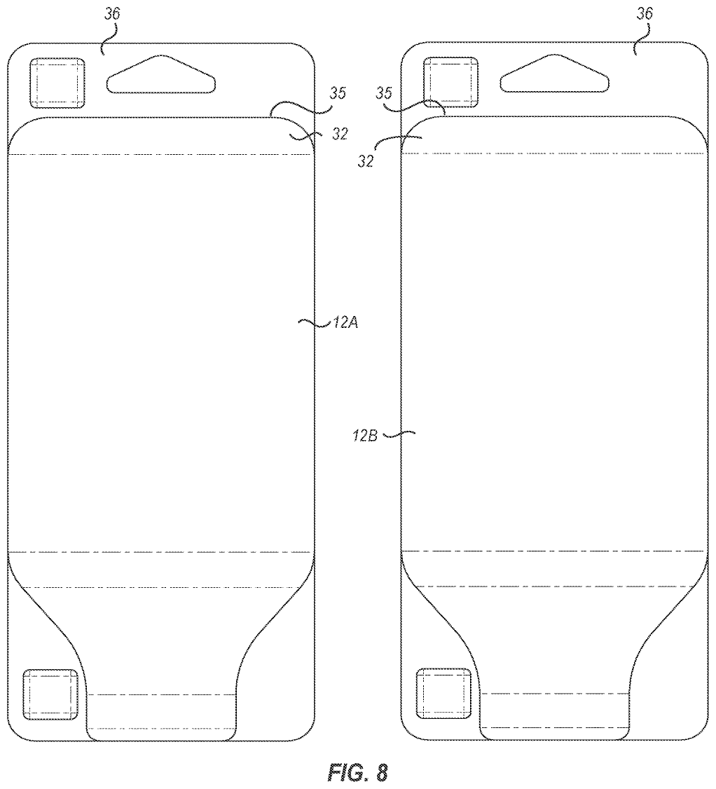

In some situations, it may be necessary to administer liquid trial product 112 to a patient from vials having different configurations, i.e., different sizes and/or shapes. For example, different suppliers of liquid trial product 112 may use vials of different configurations, different types of liquid trial product 112 may be stored in vials of different configurations, or a single supplier may switch vial configurations between the production of different batches of liquid trial product 112. Other causes may also exist for the need to administer liquid trial product 112 to a patient from vials of different configurations. Although the liquid trial product 112 may be administered to a patient from vials of different configurations, in some blinded studies it may be preferred that the patient and/or the administering person not be able to discern that the vials have different configurations. To this end, blinding shells can be formed that have internal cavities of different configurations, i.e., different sizes and/or shapes, to accommodate vials of different configurations, but have exterior surfaces with identical configurations. For example, depicted in FIG. 7 is a cross sectional view of blinding shell 12A in a closed position but with vial 14 removed. As shown in FIG. 7, blinding shell 12A includes cavity 18, hanging tab 36 and floor 32 that is disposed therebetween. Specifically, floor 32 extends between inside face 34 and outside face 35 (FIG. 8). For purposes of clarity, outside face 35 of floor 32 is also shown in FIG. 7 by dashed line. In the depicted embodiment, floor 32 extending between faces 34 and 35 is solid, i.e., no openings or pockets are formed therein. However, in other embodiments, one or more pockets or openings can be formed into floor 32 from inside face 34 so as to decrease material costs.

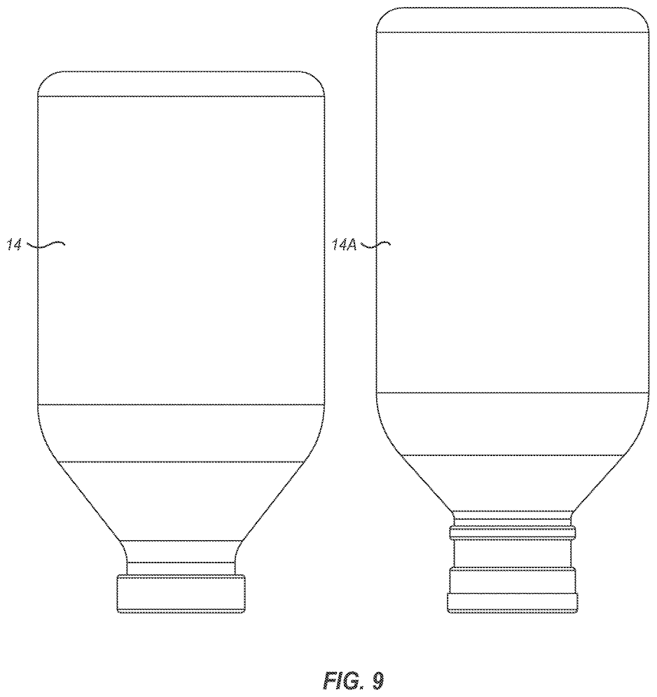

Also shown in FIG. 7 is a cross sectional view of an alternative blinding shell 12B in a closed position but with the vial removed therefrom Like elements between blinding shell 12B and blinding shell 12A are identified by like reference characters. Blinding shell 12B is identical to blinding shell 12A except that the length of cavity 18 of blinding shell 12B has been lengthened relative to cavity 18 of binding shell 12A by decreasing the thickness of floor 32. That is, the length of cavity 18 of blinding shell 12B has been lengthened by forming inside face 34 of floor 32 closer toward outside face 35 but not adjusting the position of outside face 35. Because cavity 18 of blinding shell 12B has been lengthened, the new cavity 18 can now receive a longer vial relative to what can be received within shell portion 54A. For example, depicted in FIG. 9 is vial 14 configured to be received within cavity 18 of blinding shell 12A. Also shown in FIG. 9 is a vial 14A that is configured to be received within cavity 18 of blinding shell 12B. Vial 14A has a different configuration than vial 14, i.e., vial 14A is longer than vial 14.

However, from the outside, as depicted in FIG. 8, blinding shells 12A and 12B have identical configurations and thus look identical. Thus, because the entire exterior surfaces of blinding shells 12A and 12B are identical, those administering and those receiving liquid trial product 112 from vials 14 and 14A disposed within blinding shells 12A and 12B are unable to discern that vials 14 and 14A have different configurations, thereby further helping to maintain the fidelity and efficacy of the blinded study.

Although FIGS. 7 and 8 show adjusting the size of cavity 18 by adjusting the thickness of floor 32, in other embodiments, the positioning of other interior surfaces or combination of surfaces can be adjusted, i.e., moved inward or outward or have a contour thereof modified, so as to modify the configuration of cavity 18 and especially to modify the diameter or width of cavity 18. For example, the interior surface of shield wall 28, body 44, and/or neck 46 can be moved or adjusted to modify the configuration of the cavity without modifying the corresponding exterior surfaces of the blinding shell. In one specific example, if the diameter or width of vail 14/14A is smaller than the diameter or width of cavity 18, sidewall 22 can be thickened along the full height of cavity 18 so as to correspond to the configuration of vail 14/14A. Alternatively, sidewall 22 can be thickened at two or more spaced apart locations along the height of cavity 18 so that vail 14/14A is sufficiently supported within cavity 18.

In view of the above, in one embodiment a kit can be provided that includes blinding shell 12A having cavity 18 and vial 14 disposed therein and that also includes blinding shell 12B having cavity 18 and vial 14A disposed therein. In the kit, cavities 18 and vials 14 and 14A of blinding shells 12A and 12B each have different configuration. However, the exteriors of blinding shells 12A and 12B are identical. The kit can also include at least 3, 4 or more additional blinding shells having a cavity with a vial disposed therein wherein the cavities and vials can be the same or different configurations. Again, however, the exteriors of each of the blinding shells are identical. The kit can be shipped or transported to a patient, medical facility or administering person for dispensing the liquid trial product 112 contained within the vials to a patient as part of a blinded study. Where the blinding shells and vials are being shipped in a disassembled state, the kit can comprise two or more blinding shells having cavities of different configurations and two or more vials having different configurations wherein the vials are configured to be received within the cavities of corresponding blinding shells and wherein the exterior of all of the blinding shells have the same configuration.

A method is also provided that includes inserting vial 14 within cavity 18 of blinding shell 12A and inserting vial 14A within cavity 18 of blinding shell 12B where the cavities and vials are of different configurations. However, the exteriors of blinding shells 12A and 12B are identical. At least 3, 4, or more additional vials can be inserted within blinding shells where the cavities thereof and vials are of the same or different configurations. Again, however, the exteriors of each of the blinding shells are identical. In one method, the now assembled vial blinding assemblies can now be administered to a patient as part of a blinded study. In other methods, either in combination or sequentially, blinding shells 12A and 12B with vials 14 and 14A therein can then be sent to a patient, medical facility, or administering person for dispensing the liquid trial product 112 contained within the vials to a patient as part of a blinded study. The blinding shells with vials can be sent to the same patient, medical facility or administering person or to different patients, medical facilities or administering persons.

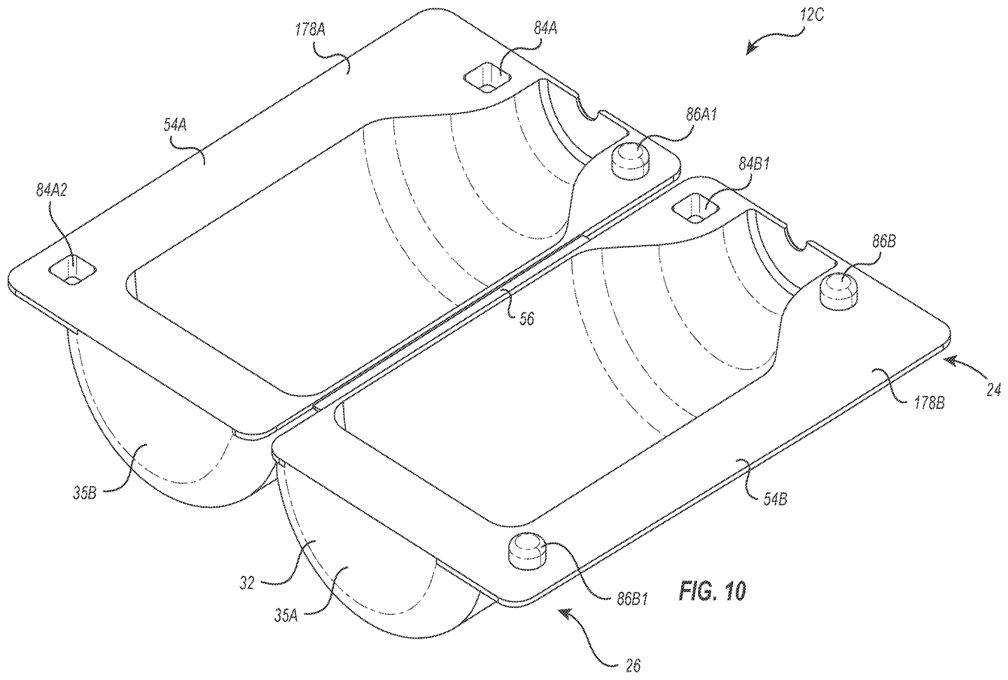

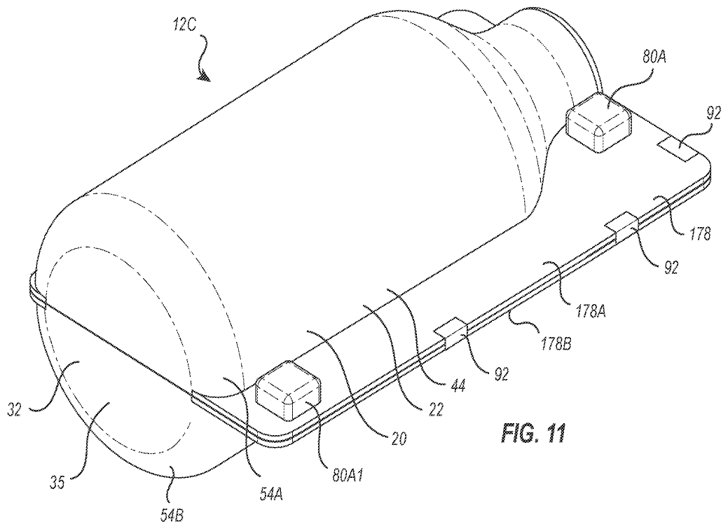

Depicted in FIGS. 10 and 11 is another alternative embodiment of a blinding shell 12C incorporating features of the present disclosure. Like element between blinding shells 12A and 12C are identified by like reference characters. As previously discussed, in some embodiments, it is not necessary to administer liquid trial product 112 intravenously and thus it is not necessary that the blinding shell be configured to support vial 14 in an inverted position. Rather, it may be more desirable, such as for ease of packaging, storing and/or administering, that the blinding shell be configured to support vial 14 in an upstanding orientation. To that end, blinding shell 12C is identical to blinding shell 12A except that hanging tab 36 has been removed so that outside face 35 of floor 32 can rest directly on a support surface, i.e., table, counter, shelf, container, etc., so as to support vial 14 therein in an upstanding orientation.

Blinding shell 12C also differs from blinding shell 12A in that closure tab portions 48A and 48B have been replaced with modified closure tab portions 178A and 178B that combine to form a closure tab 178. Closure tab portions 178A and 178B differ from closure tabs 48A and 48B by extending along the length of sidewall 22 and body 44 and also by extending outward beyond exterior surface 20 of body 44. That is, with the removal of hanging tab 36, there is no longer a mechanism for securing together second end 26 shell portions 54A and 54B of blinding shell 12C. As such, closure tab portions 178A and 178B are added and configured so that projections 86 and coupling holes 84 can be formed thereon, respectively, at second end 26. As such, as blinding shell 12C is moved to the closed position so as to enclose a vial 14 therein, the opposing ends of blinding shell 12C can be secured together using the press fit connections as previously discussed. As needed, any number of projections 86 and coupling holes 84 can be formed along the length of closure tab portions 178A and 178B. Again, once blinding shell 12C is moved to the closed position, one or more seals 92 can be applied that extend between shell portions 54A and 54B, such as between closure tab portions 178A and 178B. All previously discussed alternatives for projections 86 and coupling holes 84 discussed above with regard to blinding shell 12A are also applicable to blinding shells 12B and 12C.

It is appreciated that vial blinding assemblies and blinding shells, along the alternatives thereof and the components thereof, as discussed herein, have unique and beneficial advantages. For example, the blinding shells are easy and relatively inexpensive to produce and can be securely attached with minimal effort. Furthermore, the blinding shells are opaque and fully cover the vial, except for the septum and potentially a small adjacent area. As such, the blinding shells are highly effective in blinded studies in that they preclude any visual exposure of the liquid trial product being tested and substantially preclude visual expose of the vial. In particular, embodiments of the blinding shell are effective at covering the retainer that secures the stopper and, more particularly, the flange or terminal end face of the retainer that encircles the aperture through which the septum of the stopper is accessed.

Embodiments of the blinding shell are also unique in that they include a hanging tab which enables easy inverted suspension of the vial for intravenous delivery of the liquid trial product. Finally, embodiments of the blinding shells can be produced with cavities of different configurations for holding vials of different configurations but have identical exterior configurations. This design helps to ensure that patients and/or those who are administering are unable to detect whether vials of different configurations are being used and thus help to improve the fidelity and efficacy of the blinded studies. Other benefits and advantages also exist.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A vial blinding kit comprising:
a first vial blinding assembly comprising:
a first blinding shell having an interior surface that bounds a first cavity and an exterior surface; and
a first vial having a chamber with a liquid trial product disposed therein, the first vial being disposed within the first cavity of the first blinding shell;
a second vial blinding assembly comprising:
a second blinding shell having an interior surface that bounds a second cavity and an exterior surface; and
a second vial having a chamber with a liquid trial product disposed therein, the second vial being disposed within the second cavity of the second blinding shell;
wherein the first cavity of the first blinding shell has a different configuration than the second cavity of the second blinding shell but the exterior surface of the first blinding shell has the same configuration as the exterior surface of the second blinding shell.

2. The vial blinding kit of claim 1, wherein the interior surface bounding the first cavity of the first blinding shell and the second cavity of the second blinding shell is at least partially formed by a floor extending between an inside face and an opposing outside face.

3. The vial blinding kit of claim 2, wherein the different configuration between the first cavity of the first blinding shell and the second cavity of the second blinding shell comprises the second cavity having a longer length than the first cavity.

4. The vial blinding kit of claim 3, wherein the longer length of the second cavity comprises the disposition between the inside face and the outside face of the second blinding shell being closer than the disposition between the inside face and the outside face of the first blinding shell.

5. The vial blinding kit of claim 3, wherein the second vial has a longer length than the first vial.

6. The vial blinding kit of claim 2, wherein the outside face of the first blinding shell is disposed in the same position as the outside face of the second blinding shell.

7. A vial blinding kit comprising:
a first vial blinding assembly comprising:
a first vial comprising a first vial configuration;
a first blinding shell having an interior surface that bounds a first cavity and an exterior surface, the first cavity of the first blinding shell configured to have a complementary size with the first vial configuration of the first vial;
a second vial blinding assembly comprising:
a second vial comprising a second vial configuration that is different than the first vial configuration of the first vial; and
a second blinding shell having an interior surface that bounds a second cavity and an exterior surface having the same configuration as the exterior surface of the first blinding shell, the second cavity of the second blinding shell configured complementary size with the second vial configuration of the second vial.

8. The vial blinding kit of claim 7, wherein the second vial configuration comprises a different size than the first vial configuration.

9. The vial blinding kit of claim 7, wherein the second vial configuration comprises a different shape than the first vial configuration.

10. The vial blinding kit of claim 7, wherein the complementary size between the first vial and the first cavity and the second vial and the second cavity is configured to enable the first vial and the second vial to longitudinally move less than 10 millimeters within the respective first cavity and second cavity.

11. The vial blinding kit of claim 7, wherein the interior surface bounding the first cavity of the first blinding shell and the second cavity of the second blinding shell is at least partially formed by a floor extending between an inside face and an opposing outside face.

12. The vial blinding kit of claim 11, wherein the floor of the second blinding shell is disposed closer to the outside face than the floor of the first blinding shell.

13. The vial blinding kit of claim 11, wherein the outside face of the first blinding shell is disposed in the same position as the outside face of the second blinding shell.

14. A vial blinding kit comprising:
a first vial blinding assembly comprising:
a first blinding shell having an interior surface that bounds a first cavity and an exterior surface, an access opening passing through the first blinding shell so as to communicate with the first cavity; and
a first vial having a chamber with a liquid trial product disposed therein, the chamber being sealed closed by a stopper having a needle penetrable septum, the first vial being disposed within the first cavity of the first blinding shell so that the septum of the first vial is aligned with the access opening of the first blinding shell;
a second vial blinding assembly comprising:
a second blinding shell having an interior surface that bounds a second cavity and an exterior surface, an access opening passing through the second blinding shell so as to communicate with the second cavity; and
a second vial having a chamber with a liquid trial product disposed therein, the chamber being sealed closed by a stopper having a needle penetrable septum, the second vial being disposed within the second cavity of the second blinding shell so that the septum of the second vial is aligned with the access opening of the second blinding shell,
wherein the first cavity of the first blinding shell has a different configuration than the second cavity of the second blinding shell but the exterior surface of the first blinding shell has the same configuration as the exterior surface of the second blinding shell.

15. The vial blinding kit of claim 14, wherein the first blinding shell and the second blinding shell are opaque.

16. The vial blinding kit of claim 14, wherein the first vial has a different configuration than the second vial.

17. The vial blinding kit of claim 16, wherein the first vial comprises a different shape than the second vial.

18. The vial blinding kit of claim 16, wherein the first cavity of the first blinding shell and the second cavity of the second blinding shell are each complementarily sized in relation to the first vial and the second vial.

19. The vial blinding kit of claim 16, wherein the interior surface bounding the first cavity of the first blinding shell has a different contour than the interior surface bounding the second cavity of the second blinding shell.

20. The vial blinding kit of claim 19, wherein the interior surface of both the first blinding shell and the second blinding shell is at least partially formed by a sidewall, and the sidewall of the first blinding shell has a different thickness than the sidewall of the second blinding shell.
